(19) United States
(12) Patent Application Publication
(10) Pub. No.: US 2020/0062789 A1
(43) Pub. Date: Feb. 27, 2020

(54) HYDROXAMIC ACID PRODRUG COMPOUND OR SALT THEREOF, LYOPHILIZED FORMULATION, LPXC INHIBITOR, AND ANTIBACTERIAL AGENT

(71) Applicant: FUJIFILM Toyama Chemical Co., Ltd., Tokyo (JP)

(72) Inventors: Muneo SHOJI, Toyama-shi (JP); Yusuke NAGATO, Toyama-shi (JP); Yuko SUZUMURA, Toyama-shi (JP)

(73) Assignee: FUJIFILM Toyama Chemical Co., Ltd., Tokyo (JP)

(21) Appl. No.: 16/482,003

(22) PCT Filed: Jan. 30, 2018
(86) PCT No.: PCT/JP2018/002874

§ 371 Date: Jul. 30, 2019

(30) Foreign Application Priority Data
Jan. 31, 2017 (JP) 2017-015108

(51) Int. Cl. C07F 9/09; A61K 31/661; A61P 31/04; A61K 9/19

(52) U.S. Cl. CPC C07F 9/09; A61K 9/19; A61P 31/04; A61K 31/661

(57) ABSTRACT

An object of the present invention is to provide a compound or a salt thereof which shows strong antibacterial activity and has excellent solubility in water, and a lyophilized formulation, an LpxC inhibitor, and an antibacterial agent comprising the compound or a salt thereof. The present invention provides a compound represented by the Formula [1] or a salt thereof:

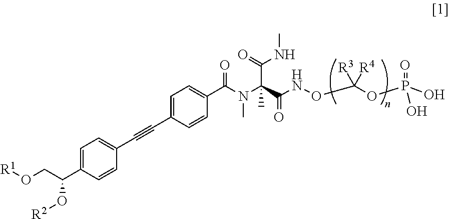

wherein R¹ represents a hydrogen atom or the like, R² represents a hydrogen atom or the like, R³ represents a hydrogen atom or the like, R⁴ represents a hydrogen atom or the like, and n represents 0 or 1.

### TECHNICAL FIELD

The present invention relates to a prodrug of (2S)-2-((4-((4-((1S)-1,2-dihydroxyethyl)phenyl)ethynyl)benzoyl)(methyl)amino)-N-hyd roxy-N′,2-dimethylmalonamide having excellent inhibitory activity against uridyldiphospho-3-O-acyl-N-acetylglucosamine deacetylase (LpxC).

### BACKGROUND ART

LpxC is an enzyme responsible for the synthesis of lipid A. Lipid A is an essential component for outer membrane formation, which is, for example, essential for the survival of gram-negative bacteria. Therefore, it is strongly expected that compounds which inhibit the activity of LpxC can be effective antibacterial agents against gram-negative bacteria including Pseudomonas aeruginosa.

For example, (2S)-2-((4-((4-((1S)-1,2-dihydroxyethyl)phenyl)ethynyl)benzoyl)(methyl)amino)-N-hyd roxy-N′,2-dimethylmalonamide (hereinafter also referred to as “compound A”) having excellent LpxC inhibitory activity is known (Patent Document 1).

In order for a drug to exert its efficacy, it is necessary for the drug to be dissolved at the absorption site. In a case in which a drug that is poorly soluble in water is orally administered, absorption from the gastrointestinal tract may not be sufficient and the drug is unlikely to exert its efficacy. In the case of parenteral administration, particularly intravenous administration, the drug needs to be administered in the dissolved form.

Hitherto, a formulation containing a compound A and a solubilizer has been known (Patent Document 2).

### PRIOR ART DOCUMENTS

### Patent Documents

Patent Document 1: WO2014/142298

Patent Document 2: WO2016/039433

### SUMMARY OF INVENTION

### Object to be Solved by the Invention

An object of the present invention is to provide a compound or a salt thereof that has excellent solubility in water and shows strong antibacterial activity against gram-negative bacteria including Pseudomonas aeruginosa and drug-resistant bacteria thereof by inhibiting LpxC, and a lyophilized formulation, an LpxC inhibitor, and an antibacterial agent comprising the compound or a salt thereof.

### Means for Solving the Object

Under these circumstances, the present inventors conducted intensive studies. As a result, the inventors found that a compound represented by the Formula [1] or a salt thereof that has excellent solubility in water and shows strong antibacterial activity against gram-negative bacteria including Pseudomonas aeruginosa and drug-resistant bacteria thereof. This has led to the completion of the present invention.

According to the present invention, the following are provided.

[1] A compound represented by the Formula [1] or a salt thereof:

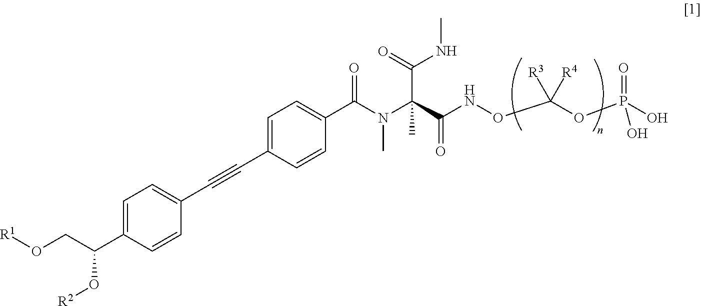

wherein R¹ represents a hydrogen atom, a group represented by the formula —P(O)(OH)₂, or a hydroxyl protecting group, R² represents a hydrogen atom, a group represented by the formula —P(O)(OH)₂, or a hydroxyl protecting group, or R¹ and R² optionally form together an optionally substituted C₁₋₃ alkylene group or a group represented by the formula —P(O)(OH)—,

R³ represents a hydrogen atom or a C₁₋₆ alkyl group,

R⁴ represents a hydrogen atom or a C₁₋₆ alkyl group, and

n represents 0 or 1.

[2] The compound or a salt thereof according to [1], wherein R¹ is a hydrogen atom.
[3] The compound or a salt thereof according to [1] or [2], wherein R² is a hydrogen atom.
[4] The compound or a salt thereof according to any one of [1] to [3], wherein R³ is a hydrogen atom or a C₁₋₃ alkyl group.
[5] The compound or a salt thereof according to any one of [1] to [4], wherein R⁴ is a hydrogen atom or a C₁₋₃ alkyl group.
[6] The compound or a salt thereof according to any one of [1] to [3], wherein n is 0.
[7] The compound or a salt thereof according to any one of [1] to [5], wherein n is 1, R³ is a hydrogen atom or a methyl group, and R⁴ is a hydrogen atom.
[8] A lyophilized formulation comprising the compound or a salt thereof according to any one of [1] to [7].
[9] An LpxC inhibitor comprising the compound or a salt thereof according to any one of [1] to [7].
[10] An antibacterial agent comprising the compound or a salt thereof according to any one of [1] to [7].

According to the present invention, the following are further provided.

[A] An injection formulation comprising: (1) the compound or a salt thereof according to any one of [1] to [6]; and (2) one, two or more selected from a sugar, a sugar alcohol, an amino acid having a hydroxyl group, and a carboxylic acid having a hydroxyl group.
[B] The injection formulation according to [A], wherein the sugar is one, two or more selected from trehalose, maltose, glucose, lactose, sucrose, fructose, dextran, and cyclodextrin,

the sugar alcohol is one, two or more selected from D-sorbitol, xylitol, inositol, isomaltose, and D-mannitol,

the carboxylic acid having a hydroxyl group is one, two or more selected from lactic acid, tartaric acid, and citric acid, and

the amino acid having a hydroxyl group is one or two selected from serine and threonine.

[C] A lyophilized formulation comprising: (1) the compound or a salt thereof according to any one of [1] to [6]; and
(2) one, two or more selected from a sugar, a sugar alcohol, an amino acid having a hydroxyl group, and a carboxylic acid having a hydroxyl group.
[D] The lyophilized formulation according to [C], wherein the sugar is one, two or more selected from trehalose, maltose, glucose, lactose, sucrose, fructose, dextran, and cyclodextrin,

the sugar alcohol is one, two or more selected from D-sorbitol, xylitol, inositol, isomaltose, and D-mannitol,

the carboxylic acid having a hydroxyl group is one, two or more selected from lactic acid, tartaric acid, and citric acid, and

the amino acid having a hydroxyl group is one or two selected from serine and threonine.

According to the present invention, the following are further provided.

[a] A method for producing a compound represented by the Formula [1a], comprising reacting a compound represented by Formula [2]:

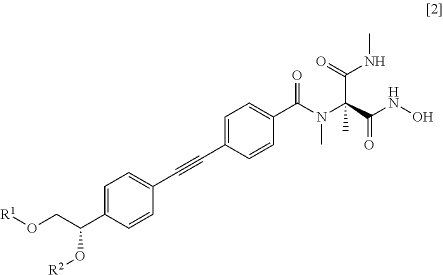

wherein R¹ and R² have the same meaning as described above, with a compound represented by Formula [3]:

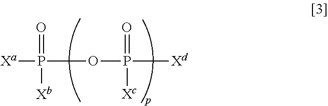

wherein X^{a} represents a halogen atom, X^{b} represents a halogen atom, X_{c} represents a halogen atom, X^{d} represents a halogen atom, and p represents 0 or 1, and then carrying out a hydrolysis reaction:

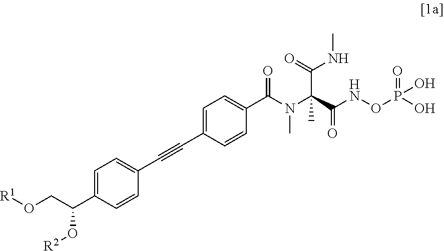

wherein R¹ and R² have the same meaning as described above.

[b] The production method according to [a], wherein R¹ is a hydrogen atom.
[c] The production method according to [a] or [b], wherein R² is a hydrogen atom.
[d] The production method according to any one of [a] to [c], wherein p is 1.
[e] The production method according to any one of [a] to [d], wherein X^{a}, X^{b}, X^{c}, and X^{d} are each a chlorine atom.

According to the present invention, the following are further provided.

<a> A method for inhibiting LpxC, comprising administering a compound represented by Formula [1] or a salt thereof to a subject:

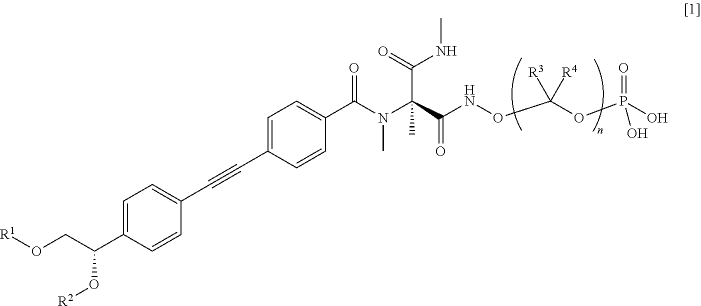

wherein R¹ represents a hydrogen atom, a group represented by the formula —P(O)(OH)₂, or a hydroxyl protecting group, R² represents a hydrogen atom, a group represented by the formula —P(O)(OH)₂, or a hydroxyl protecting group, or R¹ and R² optionally form together an optionally substituted C₁₋₃ alkylene group or a group represented by the formula —P(O)(OH)—,

R³ represents a hydrogen atom or a C₁₋₆ alkyl group,

R⁴ represents a hydrogen atom or a C₁₋₆ alkyl group, and

n represents 0 or 1.

<b> A method for controlling bacteria, comprising administering the compound represented by Formula [1] or a salt thereof to a subject.
<c> The compound represented by Formula [1] or a salt thereof for use in treatment of LpxC inhibition.
<d> The compound represented by Formula [1] or a salt thereof for use in antibacterial treatment.
<d> Use of the compound represented by Formula [1] or a salt thereof for producing an LpxC inhibitor.
<f> Use of the compound represented by Formula [1] or a salt thereof for producing an antibacterial agent.

### Advantageous Effects of Invention

The compound of the present invention shows strong antibacterial activity and has excellent solubility in water, and thus, is effective as a medicine.

### EMBODIMENT OF CARRYING OUT THE INVENTION

Hereinafter, the present invention will be described in detail.

In this description, “%” means “% by mass” unless otherwise specified.

In this description, each term has the following corresponding meaning unless otherwise specified.

The term “halogen atom” means a fluorine atom, a chlorine atom, a bromine atom, or an iodine atom.

The term “C₁₋₆ alkyl group” means, for example, a straight- or branched-chain C₁₋₆ alkyl group such as a methyl, ethyl, propyl, isopropyl, butyl, sec-butyl, isobutyl, tert-butyl, pentyl, isopentyl, or hexyl group.

The term “C₁₋₃ alkyl group” means a methyl, ethyl, propyl, or isopropyl group.

The term “C₂₋₆ alkenyl group” means, for example, a straight- or branched-chain C₂₋₆ alkenyl group such as a vinyl, allyl, propenyl, isopropenyl, butenyl, isobutenyl, 1,3-butadienyl, pentenyl, or hexenyl group.

The term “aryl group” means, for example, a phenyl or naphthyl group.

The term “ar(C₁₋₆)alkyl group” means, for example, an ar(C₁₋₆)alkyl group such as a benzyl, diphenylmethyl, trityl, phenethyl, or naphthyl methyl group.

The term “C₁₋₃ alkylene group” means a methylene, ethylene, or propylene group.

The term “C₁₋₆ alkoxy group” means, for example, a straight- or branched-chain C₁₋₆ alkyloxy group such as a methoxy, ethoxy, propoxy, isopropoxy, butoxy, isobutoxy, sec-butoxy, tert-butoxy, pentyloxy, or hexyloxy group.

The term “C₁₋₆ alkoxy C₁₋₆ alkyl group” means, for example, a C₁₋₆ alkyloxy C₁₋₆ alkyl group such as a methoxy methyl or 1-ethoxyethyl group.

The term “C₂₋₁₂ alkanoyl group” means, for example, a straight- or branched-chain C₂₋₁₂ alkanoyl group such as an acetyl, propionyl, valeryl, isovaleryl, or pivaloyl group.

The term “aroyl group” means, for example, a benzoyl or naphthoyl group.

The term “acyl group” means, for example, a formyl group, a succinyl group, a glutaryl group, a maleoyl group, a phthaloyl group, a C₂₋₁₂ alkanoyl group, or an aroyl group.

The term “C₁₋₆ alkoxy carbonyl group” means, for example, a straight- or branched-chain C₁₋₆ alkyloxy carbonyl group such as a methoxy carbonyl, ethoxy carbonyl, isopropoxy carbonyl, tert-butoxy carbonyl, or 1,1-dimethylpropoxy carbonyl group.

The term “ar(C₁₋₆)alkoxy carbonyl group” means, for example, an ar(C₁₋₆)alkyloxy carbonyl group such as a benzyloxy carbonyl or phenethyloxy carbonyl group.

The term “aryloxy carbonyl group” means, for example, a phenyloxy carbonyl or naphthyloxy carbonyl group.

The term “C₁₋₆ alkylsulfonyl group” means, for example, a C₁₋₆ alkylsulfonyl group such as a methylsulfonyl, ethylsulfonyl, or propylsulfonyl group.

The term “arylsulfonyl group” means, for example, a benzene sulfonyl, p-toluene sulfonyl, or naphthalene sulfonyl group.

The term “silyl group” means, for example, a trimethylsilyl, triethylsilyl, or tributylsilyl group.

Hydroxyl protecting groups include all groups that can be used as a usual protecting group for a hydroxyl group, which are, for example, the groups described in W. Greene et al., Protective Groups in Organic Synthesis, the 4th edition, pp. 16 to 299, 2007, John Wiley & Sons, INC. Specific examples thereof include a C₁₋₆ alkyl group, a C₂₋₆ alkenyl group, an ar(C₁₋₆)alkyl group, an C₁₋₆ alkoxy C₁₋₆ alkyl group, an acyl group, a C₁₋₆ alkoxy carbonyl group, an ar(C₁₋₆)alkoxy carbonyl group, a C₁₋₆ alkylsulfonyl group, an arylsulfonyl group, a silyl group, a tetrahydrofuranyl group, and a tetrahydropyranyl group. These groups may be substituted by one or more groups selected from Substituent Group A.

Amino protecting groups include all groups that can be used as a usual protecting group for an amino group, which are, for example, the groups described in W. Greene et al., Protective Groups in Organic Synthesis, the 4th edition, pp. 696 to 926, 2007, John Wiley & Sons, INC. Specific examples thereof include an ar(C₁₋₆)alkyl group, a C₁₋₆ alkoxy C₁₋₆ alkyl group, an acyl group, a C₁₋₆ alkoxy carbonyl group, an ar(C₁₋₆)alkoxy carbonyl group, an aryloxy carbonyl group, a C₁₋₆ alkylsulfonyl group, an arylsulfonyl group, and a silyl group. These groups may be substituted by one or more groups selected from Substituent Group A.

Carboxyl protecting groups include all groups that can be used as a usual protecting group for a carboxyl group, which are, for example, the groups described in W. Greene et al., Protective Groups in Organic Synthesis, the 4th edition, pp. 533 to 643, 2007, John Wiley & Sons, INC. Specific examples thereof include C₁₋₆ alkyl group, a C₁₋₆ alkenyl group, an ar(C₁₋₆)alkyl group, a C₁₋₆ alkoxy C₁-₆ alkyl group, and a silyl group. These groups may be substituted by one or more groups selected from Substituent Group A.

Phosphoric acid protecting groups include all groups that can be used as a usual protecting group for a phosphoric acid group, which are, for example, the groups described in W. Greene et al., Protective Groups in Organic Synthesis, the 4th edition, pp. 934 to 985, 2007, John Wiley & Sons, INC. Specific examples thereof include a C₁₋₆ alkyl group, an aryl group, and an ar(C₁₋₆)alkyl group. These groups may be substituted by one or more groups selected from Substituent Group A.

Aliphatic hydrocarbons include pentane, hexane, cyclohexane, and decahydronaphthalene.

Halogenated hydrocarbons include methylene chloride, chloroform, and dichloroethane.

Alcohols include methanol, ethanol, propanol, 2-propanol, butanol, and 2-methyl-2-propanol.

Ethers include diethyl ether, diisopropyl ether, dioxane, tetrahydrofuran, anisole, ethylene glycol dimethyl ether, diethylene glycol dimethyl ether, and diethylene glycol diethyl ether.

Ketones include acetone, 2-butanone and 4-methyl-2-pentanone.

Esters include methyl acetate, ethyl acetate, propyl acetate, and butyl acetate.

Amides include N,N-dimethyl formamide, N,N-dimethyl acetamide, and 1-methyl-2-pyrrolidone.

Nitriles include acetonitrile and propionitrile.

Aromatic hydrocarbons include benzene, toluene, and xylene.

The substituent group used herein has the following meaning.

Substituent Group A: halogen atom, cyano group, nitro group, C₁₋₆ alkyl group, aryl group, C₁₋₆ alkoxy group, oxo group

Compounds preferable as the compound of the present invention include the following compounds.

R¹ is a hydrogen atom, a group represented by the formula —P(O)(OH)₂, or a hydroxyl protecting group.

A compound in which R¹ is a hydrogen atom or a group represented by the formula —P(O)(OH)₂ is preferable, and a compound in which R¹ is a hydrogen atom is more preferable.

R² is a hydrogen atom, a group represented by the formula —P(O)(OH)₂, or a hydroxyl protecting group.

A compound in which R² is a hydrogen atom or a group represented by the formula —P(O)(OH)₂ is preferable, and a compound in which R² is a hydrogen atom is more preferable.

A compound in which R¹ is a hydrogen atom and R² is a hydrogen atom is preferable.

In another embodiment, R¹ and R² form together an optionally substituted C₁₋₃ alkylene group or a group represented by the formula —P(O)(OH)—.

A C₁₋₃ alkylene group formed by R¹ and R² may be substituted by one or more groups selected from Substituent Group A.

A compound in which R¹ and R² form together a group represented by the formula —P(O)(OH)— is preferable.

R³ is a hydrogen atom or C₁₋₆ alkyl.

A compound in which R³ is a hydrogen atom or a C₁₋₃ alkyl group is preferable, and a compound in which R³ is a hydrogen atom or a methyl group is more preferable.

R⁴ is a hydrogen atom or C₁₋₆ alkyl.

A compound in which R⁴ is a hydrogen atom or a C₁₋₃ alkyl group is preferable, a compound in which R⁴ is a hydrogen atom or a methyl group is more preferable, and a compound in which R⁴ is a hydrogen atom is still more preferable.

A compound in which R³ is a hydrogen atom or a methyl group and R⁴ is a hydrogen atom or a methyl group is preferable, and a compound in which R³ is a hydrogen atom or a methyl group and R⁴ is a hydrogen atom is more preferable.

n is 0 or 1.

A compound in which n is 0 is preferable.

In another embodiment, the compound of the present invention is preferably a compound represented by the Formula [1a]:

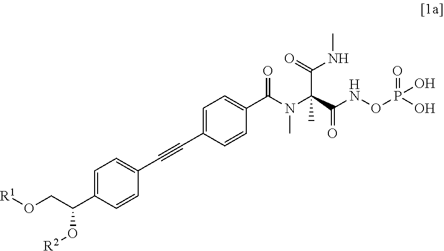

wherein R¹ and R² have the same meaning as described above. Preferable ranges of R¹ and R² are the same as described above.

The compounds listed in the table below are preferable as the compound of the present invention.

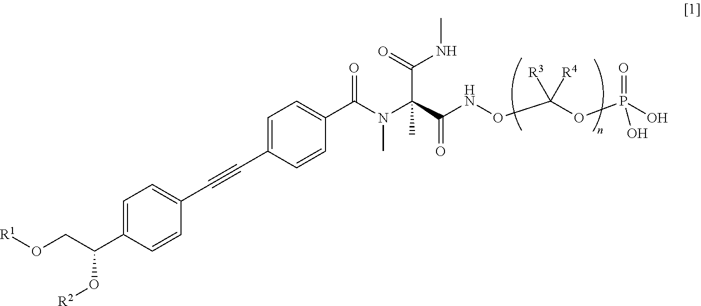

TABLE 1

| R¹ | R² | n | R³ | R⁴ |
|---|---|---|---|---|
| H | H | 0 | — | — |
| H | H | 1 | H | H |
| H | H | 1 | CH₃ | H |
| P(O)(OH)₂ | H | 0 | — | — |

| | | | |
|---|---|---|---|
| —P(O)(OH)— | 0 | — | — |

Usually known salts of phosphoric acid can be mentioned as a salt of the compound of Formula [1].

Examples of salts of phosphoric acid include: salts with alkaline metals such as sodium and potassium; salts with alkaline earth metals such as calcium and magnesium; ammonium salts; and salts with nitrogenous organic bases such as trimethylamine, triethylamine, tributylamine, pyridine, N,N-dimethyl aniline, N-methyl piperidine, N-methyl morpholine, diethyl amine, dicyclohexylamine, procaine, dibenzylamine, N-benzyl-β-phenethylamine, 1-ephenamine, and N,N′-dibenzylethylenediamine.

Among the above-described salts, preferable salts include pharmacologically acceptable salts.

In a case in which there are isomers (for example, optical isomers, geometric isomers, and tautomers) of the compound of Formula [1] or a salt thereof, the present invention includes these isomers and also includes solvates, hydrates, and crystals of various forms.

The compound of the present invention may be combined with one, two or more pharmaceutically acceptable carriers, excipients, or diluents to form a pharmaceutical formulation.

Examples of carriers, excipients, and diluents include water, lactose, dextrose, fructose, sucrose, sorbitol, mannitol, polyethylene glycol, propylene glycol, starch, gum, gelatin, alginate, calcium silicate, calcium phosphate, cellulose, water syrup, methyl cellulose, polyvinyl pyrrolidone, alkyl parahydroxy benzosorbate, talc, magnesium stearate, stearic acid, glycerin, and various oils such as sesame oil, olive oil and soybean oil.

In addition, if necessary, additives such as commonly used fillers, binders, disintegrants, pH adjusters, and solubilizers may be mixed with the above-described carriers, excipients, or diluents such that oral or parenteral medicines such as tablets, pills, capsules, granules, powders, solutions, emulsions, suspensions, ointments, injections, and skin patches can be prepared by conventional formulation techniques.

The administration method, dose, and administration frequency of the compound of the present invention can be appropriately selected in accordance with the patient's age, weight, and conditions. In general, for adults, 0.01 to 1000 mg/kg per day may be given in a single dose or divided doses by oral or parenteral administration (for example, administration by injection or infusion, or rectal administration).

The compound of the present invention is preferably administered as an injection.

A pharmaceutical composition comprising the compound of the present invention is provided as preferably a solution, a frozen solution, or a lyophilized formulation, and more preferably a lyophilized formulation.

Next, the method for producing the compound of the present invention will be described.

The compound of the present invention is produced by combining methods known per se, but can be produced, for example, according to the production methods described below.

### Production Method 1

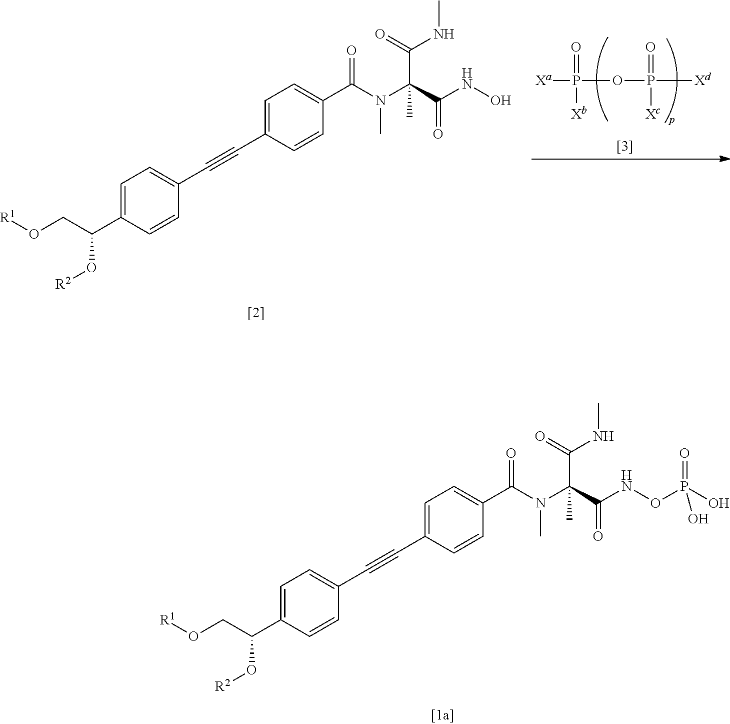

wherein R¹, R², X^{a}, X^{b}, X^{c}, X^{d}, and p have the same meaning as described above.

Examples of a compound represented by Formula [2] include (S)-2-(4((4((S)-2,2-dimethyl-1,3-dioxolan-4-yl)phenypethynye-N-methylbenzamide)-N′-hydroxy-N³,2-dimethylmalonamide.

Examples of a compound represented by Formula [3] include oxyphosphorus chloride and diphosphoryl chloride.

A compound represented by Formula [1a] can be produced by reacting a compound represented by Formula [2] with a compound represented by Formula [3] under the presence of a base and subjecting the resulting product to a hydrolysis reaction.

(1-1) Reaction of the Compound represented by Formula [2] and the Compound Represented by Formula [3]

A solvent used in this reaction is not particularly limited as long as it does not adversely affect the reaction. Examples thereof include aliphatic hydrocarbons, halogenated hydrocarbons, alcohols, ethers, ketones, esters, amides, nitriles, aromatic hydrocarbons, dimethyl sulfoxide, and water, which may be mixed for use. Preferable solvents include ethers.

The amount of the solvent used may be 1 to 50 times (v/w) and preferably 2 to 10 times (v/w) the amount of the compound represented by Formula [2].

A base used for this reaction can be an organic base which is, for example, pyridine.

The amount of the base used may be 1 to 50 times moles and preferably 1 to 5 times moles of the compound represented by Formula [2].

This reaction may be carried out at −50° C. to 100° C. and preferably −30° C. to 30° C. for 30 minutes to 12 hours.

### (1-2) Hydrolysis Reaction

A solvent used in this reaction is not particularly limited as long as it does not adversely affect the reaction. Examples thereof include aliphatic hydrocarbons, halogenated hydrocarbons, alcohols, ethers, ketones, esters, amides, nitriles, aromatic hydrocarbons, dimethyl sulfoxide, and water, which may be mixed for use. Preferable solvents include ethers.

The amount of a solvent used may be 1 to 50 times (v/w) and preferably 2 to 10 times (v/w) the amount of the compound represented by Formula [2].

The hydrolysis reaction is preferably a hydrolysis reaction using an acid.

An acid used for this reaction can be an inorganic acid which is, for example, hydrochloric acid.

The amount of the acid used may be 1 to 50 times moles and preferably 1 to 5 times moles of the compound represented by Formula [2].

This reaction may be carried out at −50° C. to 100° C. and preferably −30° C. to 30° C. for 30 minutes to 12 hours.

### Production Method 2

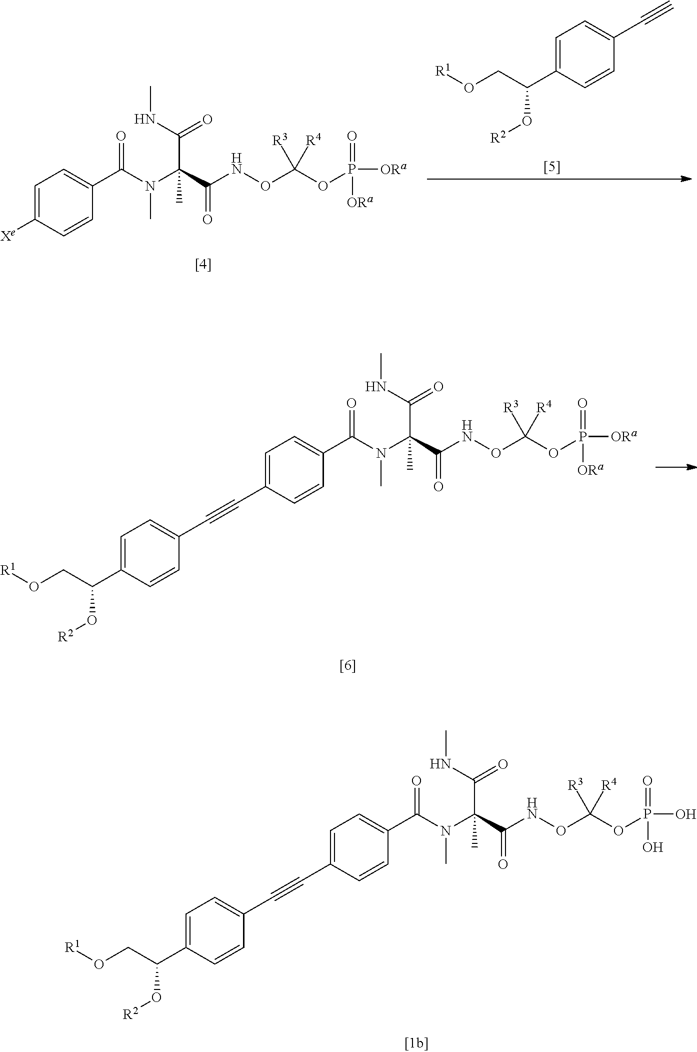

wherein R^{a} represents a phosphoric acid protecting group, X^{e} represents a bromine atom or an iodine atom, and R¹, R², R³, and R⁴ have the same meaning as described above.

Examples of a compound represented by Formula [4] include phosphoric acid di-tert-butyl((((2S)-2-((4-chlorobenzoyl)(methyl)amino)-2-methyl-3-(methylamino)-3-o xo)propanoyl)amino)oxymethyl.

Examples of a compound represented by Formula [5] include (4S)-4-(4-ethynylphenyl)-2,2-dimethyl-1,3 -dioxolane.

(2-1)

A compound represented by Formula [6] can be produced by reacting a compound represented by Formula [5] with a compound represented by Formula [4] under the presence or absence of a base, the presence or absence of a copper catalyst, the presence or absence of a ligand, and the presence of a palladium catalyst.

This reaction may be carried out by the method described in WO2011/132712 or the like or a method according thereto.

A solvent used in this reaction is not particularly limited as long as it does not adversely affect the reaction. Examples thereof include aliphatic hydrocarbons, halogenated hydrocarbons, alcohols, ethers, ketones, esters, amides, aromatic hydrocarbons, dimethyl sulfoxide, and water, which may be mixed for use. Preferable solvents include ethers.

Examples of a base optionally used in this reaction include: organic bases such as sodium methoxide, sodium ethoxide, potassium tert-butoxide, pyridine, dimethylaminopyridine, and triethylamine; and inorganic bases such as sodium hydride, sodium hydroxide, potassium hydroxide, sodium hydrogen carbonate, potassium carbonate, and sodium carbonate. Preferable bases include triethylamine.

The amount of the base used may be 1 to 50 times moles and preferably 1 to 10 times moles of the compound represented by Formula [4].

Examples of a copper catalyst optionally used in this reaction include copper bromide and copper iodide.

The amount of the copper catalyst used may be 0.01 to 50 times moles and preferably 0.1 to 5 times moles of the compound represented by Formula [4].

Examples of a ligand optionally used in this reaction include tri-t-butyl phosphine, tricyclohexyl phosphine, triphenyl phosphine, tritolyl phosphine, tributyl phosphite, tricyclohexyl phosphite, triphenyl phosphite, 1,1′-bis(diphenylphosphino)ferrocene, 2,2′-bis(diphenylphosphino)-1,1′-binaphthyl, 2-dicyclohexylphosphino-2′,6′-dimethoxybiphenyl, 2-dicyclohexylphosphino-2′,4′,6′-triisopropylbiphenyl, 2-(di-t-butylphosphino)-2′,4′,6′-triisopropylbiphenyl, and 2-(di-t-butylphosphino)biphenyl, which may be combined for use.

The amount of the ligand used may be 0.00001 to 1 times moles and preferably 0.001 to 0.1 times moles of the compound represented by Formula [4].

Examples of a palladium catalyst used in this reaction include: metallic palladium such as palladium-carbon or palladium black; inorganic palladium salts such as palladium chloride and palladium(II) sodium chloride trihydrate; organopalladium salts such as palladium acetate; organopalladium complexes such as tetrakis(triphenylphosphine)palladium(0), bis(triphenylphosphine)palladium(II) dichloride, bis(acetonitrile)palladium(II) dichloride, bis(benzonitrile)palladium(II) dichloride, 1,1′-bis(diphenylphosphino)ferroocenepalladium(II) dichloride, tris(dibenzylideneacetone)dipalladium(0), bis(dibenzylideneacetone)palladium(0), bis(tricyclohexylphosphine)palladium(II) dichloride, bis(tri-o-tolylphosphine)palladium(II) dichloride, bis(tri-t-butylphosphine)palladium(II) dichloride, (1,3-bis(2,6-diisopropylphenyl)imidazolidinylidene)(3-chloro-pyridyl)palladium(II) dichloride, and bis(di-tert-butyl(4-dimethylaminophenyl)phosphine)palladium(II)dichloride; and polymer-immobilized organopalladium complexes such as polymer-supported bis(acetate)triphenylphosphinepalladium(II) and polymer-supported di(acetate)dicyclohexylphenylphosphinepalladium(II), which may be combined for use.

The amount of the palladium catalyst used may be 0.00001 to 1 times moles and preferably 0.001 to 0.1 times moles of the compound represented by Formula [4].

The amount of the compound represented by Formula [5] used may be 1 to 50 times moles and preferably 1 to 5 times moles of the compound represented by Formula [4].

This reaction may be carried out at −50° C. to 200° C. and preferably −10° C. to 50° C. for 10 minutes to 48 hours.

This reaction may be carried out preferably under an inert gas (for example, nitrogen or argon) atmosphere.

(2-2)

A compound represented by Formula [1b] can be produced by deprotecting a compound represented by Formula [6].

A deprotection reaction can be carried out by, for example, the method described in Greene's Protective Groups in Organic Synthesis, the 5th edition, pp. 1203 to 1262, 2014, John Wiley & Sons, INC.

Next, a method for producing the compound of Formula [4], which is a starting material for producing the compound of the present invention, will be described.

### Production Example A

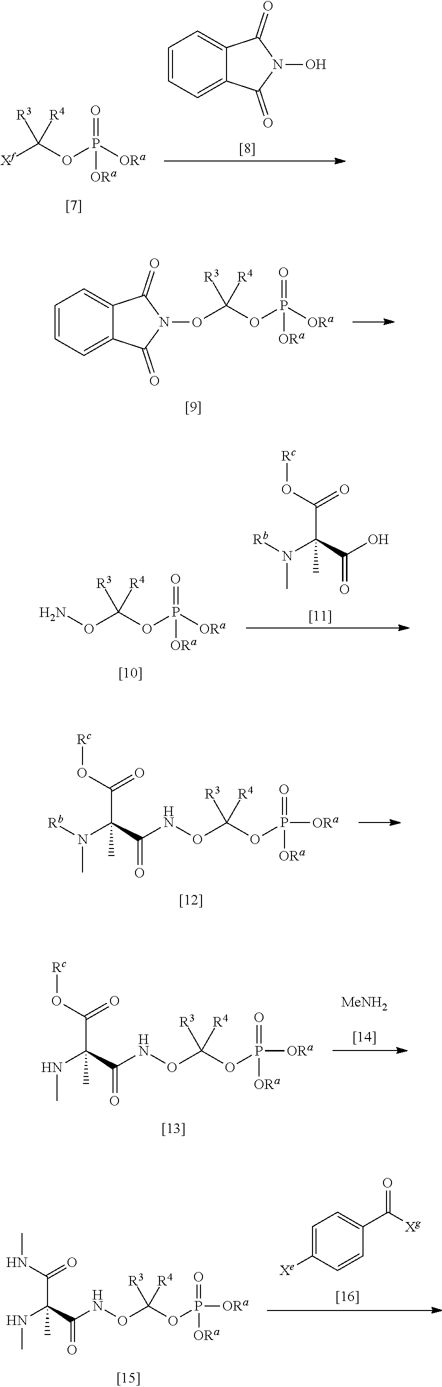

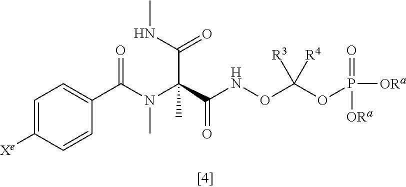

wherein R^{b} represents an amino protecting group, R^{c} represents a carboxyl protecting group, X^{f} represents a halogen atom, X^{g} represents a halogen atom, R¹, R², R³, R⁴, R^{a}, and X^{e} have the same meaning as described above.

### (A-1)

Examples of a compound represented by Formula [7] include phosphoric acid di-tert-butylchloromethyl.

A compound represented by Formula [10] can be produced by reacting a compound represented by Formula [7] with N-hydroxyphthalimide and deprotecting the resulting product. This reaction can be produced, for example, according to the method described in the Fourth Series of Experimental Chemistry, volume 20, pp. 344 to 345, 1992, Maruzen or a method according thereto.

### (A-2)

Examples of a compound represented by Formula [11] include (2R)-2-(benzyloxy carbonyl(methyl)amino)-3-ethoxy-2-methyl-3-oxopropionic acid.

A compound represented by Formula [12] can be produced by reacting a compound represented by Formula [10] with a compound represented by Formula [11] under the presence of a condensing agent and the presence or absence of a base. This reaction may be produced by the method described in, for example, WO2011/132712 or a method according thereto.

### (A-3)

A compound represented by Formula [13] can be produced by deprotecting a compound represented by Formula [12]. This reaction can be carried out by, for example, the method described in Protective Groups in Organic Synthesis, the 4th edition, pp. 16 to 299, 2007, John Wiley & Sons, INC.

### (A-4)

A compound represented by Formula [15] can be produced by reacting a compound represented by Formula [13] with methylamine under the presence of a condensing agent and the presence or absence of a base. This reaction may be produced by the method described in, for example, WO2011/132712 or a method according thereto.

### (A-5)

Examples of a compound represented by Formula [16] include 4-iodobenzoyl chloride.

A compound represented by Formula [4] can be produced by reacting a compound represented by Formula [16] with a compound represented by Formula [15] under the presence or absence of a base. This reaction may be produced by the method described in, for example, WO2011/132712 or a method according thereto.

Among the compounds used in the above-described production methods, compounds having a substituent group which can be protected, for example, an amino group, a hydroxyl group, or a carboxyl group, are protected in advance by protecting these groups with a usual protecting group. After the reaction, these protecting groups can also be removed by a method known per se.

### Production Method 3

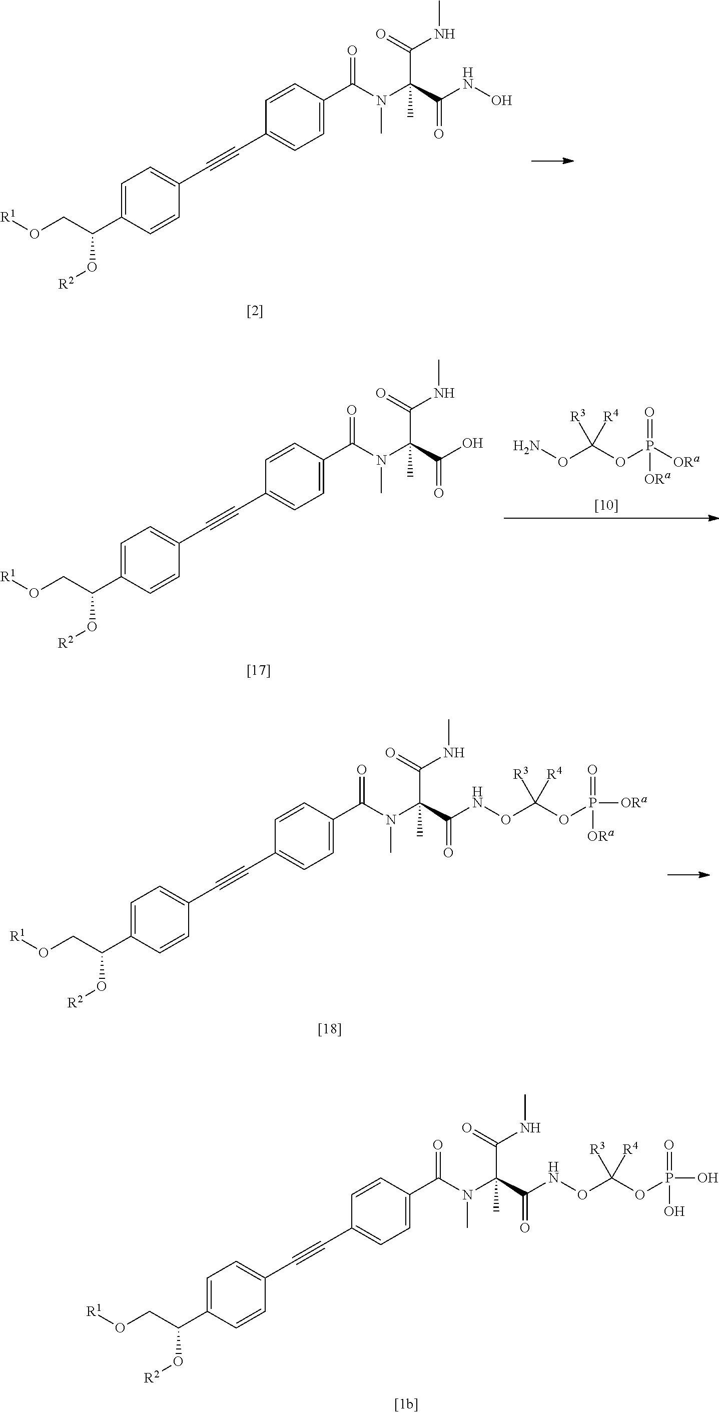

wherein R¹, R²R³, R⁴, and Ra have the same meaning as described above.

Examples of a compound represented by Formula [2] include (S)-2-(4-((4-((S)-2,2-dimethyl-1,3-dioxolan-4-yl)phenyl)ethynyl)-N-methylbenzamide)-N′-hydroxy-N³,2-dimethylmalonamide.

(3-1)

A compound represented by Formula [17] can be produced by subjecting a compound represented by Formula [2] to a hydrolysis reaction.

A solvent used in this reaction is not particularly limited as long as it does not adversely affect the reaction. Examples thereof include aliphatic hydrocarbons, halogenated hydrocarbons, alcohols, ethers, ketones, esters, amides, nitriles, aromatic hydrocarbons, dimethyl sulfoxide, and water, which may be mixed for use. Preferable solvents include nitriles.

The amount of the solvent used may be 1 to 50 times (v/w) and preferably 2 to 10 times (v/w) the amount of the compound represented by Formula [2].

The hydrolysis reaction is preferably a hydrolysis reaction using an acid.

An acid used for this reaction can be an inorganic acid which is, for example, hydrochloric acid.

The amount of the acid used may be 1 to 50 times moles and preferably 1 to 5 times moles of the compound represented by Formula [2].

This reaction may be carried out at −50° C. to 100° C. and preferably −30° C. to 50° C. for 30 minutes to 3 days.

(3-2)

A compound represented by Formula [10] can be produced by the method described in Production Example A.

A compound represented by Formula [18] can be produced by reacting a compound represented by Formula [17] with a compound represented by Formula [10] under the presence or absence of a condensing agent.

This reaction may be carried out by the method described in, for example, WO2011/132712 or a method according thereto.

(3-3)

A compound represented by Formula [1b] can be produced by deprotecting a compound represented by Formula [18]. This reaction can be carried out by, for example, the method described in Protective Groups in Organic Synthesis, the 4th edition, pp. 16 to 299, 2007, John Wiley & Sons, INC.

Next, a method for producing the pharmaceutical composition of the present

### Production Method 4 Lyophilized formulation

An aqueous solution containing the compound of the present invention can be lyophilized, thereby obtaining a lyophilized formulation.

This step may be performed according to a lyophilization method which is usually performed. For example, the step can be carried out in accordance with “15.2 Toketsu-kanso no jissai (Practice of lyophilization)” described in “Iyakuhin no jissai (Practice of pharmaceutical products),” vol. 11, Seizai no tanni sousa to kikai (Unit operation and machine for formulation), edited by Yoshinobu Nakai, pp. 388 to 396 (1988, Hirokawa-Shoten Ltd.).

Additives can be added to the lyophilized formulation of the present invention to improve solubility, appearance, or storage stability.

Examples of additives include amino acids, polyethers, sugars, sugar alcohols, salts, carboxylic acids having a hydroxyl group, urea, ethyl urea, creatinine, nicotinic acid amide, trometamol, purified soy lecithin, ovalbumin, bovine serum albumin, and polysorbate 80. These can be used alone or in combination of two or more thereof.

Examples of amino acids used as additives include glycine, L-alanine, L-phenylalanine, L-valine, L-leucine, L-isoleucine, taurine, DL-methionine, L-serine, L-threonine, L-glutamine, sodium L-glutamate, acetyl tryptophan, and L-histidine.

Examples of polyethylene glycols used as additives include polyethylene glycol 300, polyethylene glycol 400, polyethylene glycol 600, polyethylene glycol 4000, and polyethylene glycol 6000.

Examples of sugars used as additives include trehalose, maltose, glucose, lactose, sucrose, fructose, dextran, and cyclodextrin.

Examples of sugar alcohols used as additives include D-sorbitol, xylitol, inositol, isomaltose, and D-mannitol.

Examples of salts used as additives include sodium acetate, sodium lactate, L-sodium tartrate, sodium citrate, sodium salicylate, sodium benzoate, and sodium caprylate.

Examples of carboxylic acids having a hydroxyl group used as additives include lactic acid, tartaric acid, and citric acid.

Examples of preferable additives include sugars, sugar alcohols, amino acids having a hydroxyl group, and carboxylic acids having a hydroxyl group.

Examples of amino acids having a hydroxyl group include L-serine or L-threonine. Examples of more preferable additives include D-sorbitol, glucose, D-threonine, and citric acid.

In addition, if necessary, commonly used osmotic pressure regulators, pH adjusters, buffers, solubilizers, stabilizers, surfactants, soothing agents and/or preservatives, and the like may be added to the formulation of the present invention.

Examples of osmotic pressure regulators include sodium chloride, glycerin, and propylene glycol.

Examples of pH adjusters and/or buffers include: acids such as hydrochloric acid, phosphoric acid, sulfuric acid, methanesulfonic acid, acetic acid, lactic acid, maleic acid, citric acid, tartaric acid, ascorbic acid, and benzoic acid; salts such as sodium hydrogen carbonate, sodium carbonate, sodium dihydrogen phosphate, potassium dihydrogen phosphate, disodium hydrogen phosphate, dipotassium hydrogen phosphate, trisodium phosphate, disodium citrate, sodium deoxycholate, and sodium sulfite; and bases such as sodium hydroxide, trometamol, monoethanolamine, diethanolamine, triethanolamine, L-arginine, and L-lysine.

Examples of solubilizers include macrogol and purified soy lecithin.

Examples of stabilizers include sodium bisulfite, sodium pyrosulfite, potassium pyrosulfite, sodium pyrophosphate, sodium thiosulfate, sodium metasulfobenzoate, sodium formaldehyde sulfoxylate, ethylene diamine, edetate sodium, thioglycolic acid, sodium gluconate, potassium L-glutamate, L-lysine-L-glutamate, sodium chondroitin sulfate, albumin, L-aspartic acid, L-cysteine and dibutylhydroxytoluene.

Examples of surfactants include sorbitan fatty acid ester, polyoxyethylene hydrogenated castor oil, polyoxyethylene sorbitan monolaurate, polyoxyethylene polyoxypropylene glycol, and polysorbate.

Examples of soothing agents include lidocaine, procaine, meprilcaine, and benzyl alcohol.

Examples of preservatives include cresol, phenol, methyl parahydroxybenzoate, ethyl parahydroxybenzoate, benzalkonium chloride, and benzethonium chloride.

In the production of the lyophilized formulation of the present invention, sterilization may be carried out according to a commonly performed procedure.

The lyophilized formulation of the present invention can be dissolved in an injection solvent so as to be provided as an injection formulation.

The pH of the injection formulation prepared from the lyophilized formulation is preferably 3.0 to 8.0, more preferably 3.5 to 7.5, and still more preferably 4.0 to 6.5.

The content of the compound of the present invention in the injection formulation prepared from the lyophilized formulation is preferably 1 to 100 mg/mL and more preferably 2 to 50 mg/mL.

The dose of the compound of the present invention is appropriately determined in accordance with the dosing regimen, patient's age, sex, form of disease, other conditions, and the like. However, the daily dose may be usually 0.1 to 1000 mg/kg for adults.

It is preferable that no organic solvents are used for the preparation of a solution, a frozen solution, and a lyophilized formulation according to the present invention. Therefore, these formulations contain no residual solvents and are safe for the human body.

The present invention will be described in the Examples, Reference Examples, Comparative Examples, and Test Examples described below. However, the present invention is not limited thereto.

Unless otherwise specified, silica gel column chromatography is flash column chromatography, for which B.W. Silica gel BW-300 manufactured by Fuji Silysia Chemical Ltd. is used as a carrier; ODS-A manufactured by YMC Co., Ltd. is used as a carrier for reverse phase silica gel column chromatography.

The mixing ratio in the eluent is the volume ratio.

An NMR spectrum shows proton NMR, internal standards are as follows, and the δ value is shown in ppm.

Heavy chloroform (CDCl₃): tetramethylsilane (0.00 ppm)

Heavy dimethyl sulfoxide (DMSO-d6): tetramethylsilane (0.00 ppm)

Heavy methanol (CD₃OD): methanol (CH₃OH) (3.30 ppm)

Heavy water (D₂O): water (H2O) (4.65 ppm)

In the NMR spectrum, for example, the description of [1.81], 1.82(3H,s) indicates that the peak derived from each diastereomer of the diastereomeric mixture is observed as a singlet at 1.81 and 1.82, and the total proton number is 3H.

Each abbreviation has the following meaning.

ESI: electrospray ionization method
IPE: diisopropyl ether
THP: tetrahydro-2H-pyran-2-yl
s: singlet
d: doublet
dd: double doublet

### m: Multiplet

### Example 1

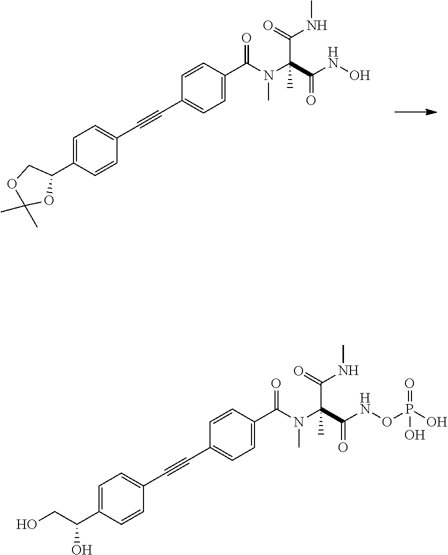 [SMILES: CNC(=O)[C@@](C)(C(=O)NO)N(C)C(=O)c1ccc(C#Cc2ccc([C@H]3COC(C)(C)O3)cc2)cc1 ; CNC(=O)[C@@](C)(C(=O)NOP(=O)(O)O)N(C)C(=O)c1ccc(C#Cc2ccc([C@H](O)CO)cc2)cc1]

Thirty-three milliliters (33 mL) of acetonitrile and 6.7 mL of pyridine were sequentially added to a reaction vessel at room temperature under a nitrogen atmosphere. Then, 10.5 g of diphosphoryl chloride was added to the reaction mixture under ice cooling. The reaction mixture was stirred for 15 minutes, and then a mixture of 10 g of (S)-2-(4-((4-((S)-2,2-dimethyl-1,3 -dioxolan-4-yl)phenyl)ethynyl)-N-methylbenzamide)-N¹-hydroxy-N³,2-dimethylmalonamide, 50 mL of acetonitrile, and 1.68 mL of pyridine was added at the same temperature. The reaction mixture was stirred for 3 hours at the same temperature and then cooled to −30° C. At the same temperature, 20 mL of water and 20 mL of concentrated hydrochloric acid were sequentially added to the reaction mixture and stirred at −15° C. for 2 hours. The reaction mixture was then cooled to −35° C. and 194 mL of a 22% aqueous sodium carbonate solution was added in one portion. While stirring the reaction mixture so as to maintain pH 7.0 to 7.5 at 10° C. or less, 5 mL of a 22% aqueous sodium carbonate solution was added three times. One hundred and thirty milliliters (130 mL) of ethyl acetate was added to the reaction mixture, the resulting solid was collected by filtration and washed with 20 mL of water, thereby obtaining a solid 1. The filtrate and the wash liquid were mixed, and the aqueous layer was separated, thereby obtaining an aqueous solution 1.

Similarly, 100 mL of acetonitrile and 20.1 mL of pyridine were sequentially added to the reaction vessel at room temperature under a nitrogen atmosphere. Under ice-cooling, 31.5 g of diphosphoryl chloride was added to the reaction mixture. The reaction mixture was stirred for 30 minutes, and then a mixture of 30 g of (S)-2-(4-((4-4(S)-2,2-dimethyl-1,3-dioxolan-4-yl)phenyl)ethynyl)-N-methylbenzamide)-N¹-hydroxy-N³,2-dimethylmalonamide, 150 mL of acetonitrile, and 5 mL of pyridine was added at the same temperature. The reaction mixture was stirred for 2 hours at the same temperature. The reaction mixture was cooled to −35° C., and 60 mL of water and 60 mL of concentrated hydrochloric acid were sequentially added. The reaction mixture was stirred at −20° C. to −15° C. for 3 hours and 30 minutes. The reaction mixture was then cooled to −40° C. and 580 mL of a 22% aqueous sodium carbonate solution was added in one portion. The reaction mixture was stirred at 2° C. for 20 minutes, and the pH was adjusted to 7.2 by adding 40 mL of a 22% aqueous sodium carbonate solution. Four hundred milliliters (400 mL) of ethyl acetate was added to the reaction mixture, the resulting solid was collected by filtration and washed with 60 mL of water, thereby obtaining a solid 2. The filtrate and the wash liquid were mixed, and the aqueous layer was separated, thereby obtaining an aqueous solution 2.

The resulting aqueous solutions 1 and 2 were then combined and acetonitrile was added. The solvent was distilled off under reduced pressure at an external bath temperature of 35° C. to 40° C. so as to adjust the liquid volume to about 250 mL. The solid obtained from the suspension was collected by filtration and washed with 40 mL of water, thereby obtaining a solid 3. The filtrate and the wash liquid were mixed, thereby obtaining an aqueous solution 3.

The solids 1, 2, and 3 were mixed, 300 mL of methanol was added, and the mixture was stirred at room temperature for 30 minutes. The mixture was filtered and the solid was washed with 40 mL of methanol. The aqueous solution 3 and acetonitrile were added to the obtained filtrate, and the solvent was distilled off under reduced pressure at an external bath temperature of 35° C. or less so as to adjust the liquid volume to about 200 mL. The obtained aqueous solution was purified by reverse phase silica gel column chromatography [eluent; acetonitrile:water=5:95] The fractions containing the desired compound were collected, and the solvent was distilled off under reduced pressure at an external bath temperature of 35° C. or less so as to adjust the liquid volume to about 400 mL. 1 mol/L hydrochloric acid was added to the obtained aqueous solution so as to adjust the pH to 7.3. The obtained aqueous solution was purified by reverse phase silica gel column chromatography [eluent:acetonitrile:water =5:5] The fractions containing the desired compound were collected, and the solvent was distilled off under reduced pressure at an external bath temperature of 35° C. or less so as to adjust the liquid volume to about 100 mL, thereby obtaining an aqueous solution 4.

Then, 4800 mL of ethanol was added to the reaction vessel, the mixture was stirred, and the aqueous solution 4 was added over 20 minutes at room temperature. The obtained mixture was stirred under ice cooling for 1 hour, and then the resulting solid was collected by filtration at the same temperature. The obtained solid was washed with 100 mL of ethanol, and then dried under reduced pressure at an external bath temperature of 10° C. for 4 hours, thereby obtaining 34.0 g of a sodium salt of (((S)-2-(4-((4-((S)-1, 2-dihydroxyethyl)phenyl)ethynyl)-N-methylbenzamide)-2-methyl-3-(methylamino)-3-oxopropanamide)oxy)phosphonic acid as a white solid.

¹H-NMR (600MHz, D₂O) δ value: 1.73 (3H, s), 2.65 (3H, s), 3.07 (3H, s), 3.58-3.64 (2H, m), 4.69 (1H, dd, J=6.6, 4.8 Hz), 7.30 (2H, d, J=8.4Hz), 7.43 (2H, d, J=8.4Hz), 7.49 (2H, d, J=8.4Hz), 7.56 (2H, d, J=7.8Hz); MS(ESI): 518[M−H]⁻

### Reference Example 1

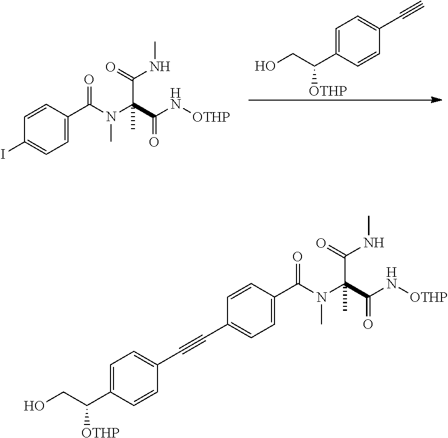

Ten milliliters (10 mL) of tetrahydrofuran was added to 1.00 g of (2S)-2-(4-iodo-N-methylbenzamide)-N ¹,2-dimethyl-N³-((tetrahydro-2H-pyran-2-yl)oxy )malonamide, 1.06 g of (2S)-2-(4-ethynylphenyl)-2-((tetrahydro-2H-pyran-2-yl)oxy)ethane-1-ol, 143 mg of dichlorobis(triphenylphosphine)palladium(II), and 77 mg of copper(I) iodide. One point one milliliters (1.1 mL) of triethylamine was added to the reaction mixture under nitrogen atmosphere with ice cooling and stirred at the same temperature for 1 hour. An aqueous saturated ammonium chloride solution and ethyl acetate were added to the reaction mixture, the pH was adjusted to 6.4 with 1 mol/L hydrochloric acid, and the organic layer was separated. The organic layer was washed with an aqueous saturated sodium chloride solution, and then 0.15 g of basic silica gel (Fuji Silysia Chemical Ltd., DNH) was added to the organic layer, and the mixture was dried over anhydrous magnesium sulfate. The insoluble matter was filtered off, and then the solvent was distilled off under reduced pressure. The obtained residue was purified by silica gel column chromatography [eluent:acetone:chloroform =50:50], thereby obtaining a brown foamy solid. Ethyl acetate and IPE were added to the obtained solid, and the solid was collected by filtration, thereby obtaining 1.08 g of (2S)-2-(4-((4-((1S)-2-hydroxy-1-((tetrahydro-2H-pyran-2-yl)oxy)ethyl)phenyl)ethynyl)-N-methylbenzamide)-N¹,2-dimethyl-N³-((tetrahydro-2H-pyran-2- yl)oxy)malonamide as a pale brown solid.

¹H-NMR (400 MHz, CDCL₃) δ value: 1.46 -1.69 (4H, m), 1.69-1.86 (9H, m), 2.11-2.14 (1H, m), [2.85]2.86 (3H, d, J=3.9Hz), [3.17]3.20 (3H, s), [3.01-3.04]3.29-3.32 (1H, m), 3.55-3.63 (1H, m), [3.55-3.63]3.85-3.90 (1H, m), 3.65-3.75 (4H, m), 4.00-4.04 (1H, m), [4.52-4.54]4.82-4.85 (1H, m), [4.73-4.76]4.90-4.92 (1H, m), [4.96]5.00 (1H, s), 7.33 (1H, d, J=8.3Hz), 7.39 (1H, d, J=8.3Hz), 7.49-7.59 (6H, m), [6.98-6.99]7.62-7.63 (1H, m), [10.10]10.50 (1H, s)

### Comparative Example 1

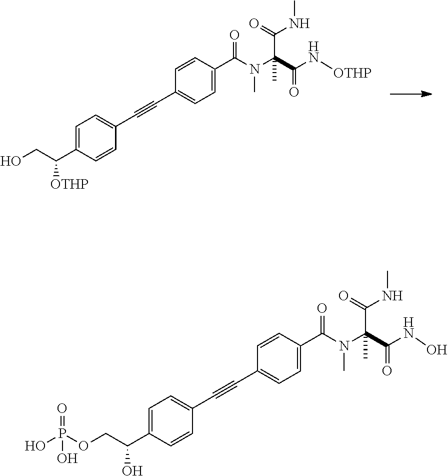

Three milliliters (3 mL) of acetonitrile and 162 μL of pyridine were added to 303 mg of (2S)-2-(4-((4-((1S)-2-hydroxy-1-((tetrahydro-2H-pyran-2-yl)oxy)ethyl)phenyl)ethynyl)-N-methylbenzamide)-N¹,2-dimethyl-N³-((tetrahydro-2H-pyran-2- yl)oxy)malonamide and stirred under ice cooling. At the same temperature, 138 μL of diphosphoryl chloride was added to the reaction mixture. The reaction mixture was stirred for 1 hour and 30 minutes under ice cooling, and then poured into a mixture of 841 mg of sodium hydrogen carbonate and 3 mL of water under ice cooling. The reaction mixture was stirred at the same temperature for 30 minutes, and then the pH was adjusted to 1 or less with concentrated hydrochloric acid. The reaction mixture was stirred at the same temperature for 1 hour and 30 minutes, and then 10 mL of ethyl acetate was added. The pH of the reaction mixture was then adjusted to 7.9 using an aqueous saturated sodium bicarbonate solution and a 20% aqueous sodium hydroxide solution. The aqueous layer was separated and concentrated to about one-third (⅓) of its original volume under reduced pressure. The obtained mixture was purified by reverse phase silica gel column chromatography [eluent:acetonitrile:water=4:96] and lyophilized, thereby obtaining 182 mg of a sodium salt of (S)-2-hydroxy-2-(4-((4-(((S)-1-(hydroxyamino)-2-methyl-3-(methylamino)-1,3-dioxopr opan-2-yl)(methyl)carbamoyl)phenyl)ethynyl)phenyl)ethyl phosphate as a pale yellow powder.

¹H-NMR (400 MHz, D₂O ) δ value: 1.66 (3H, s), 2.66 (3H, s), 3.03 (3H, s), 3.68-3.86 (2H, m), 4.80-4.83 (1H, m), 7.37 (2H, d, J=8.0 Hz), 7.42 (2H, d, J=7.8 Hz), 7.51 (2H, d, J=8.0Hz), 7.58 (2H, d, J=7.8Hz); MS (ESI): 564[M+2Na]⁺,518[M−H]⁻

### Comparative Example 2

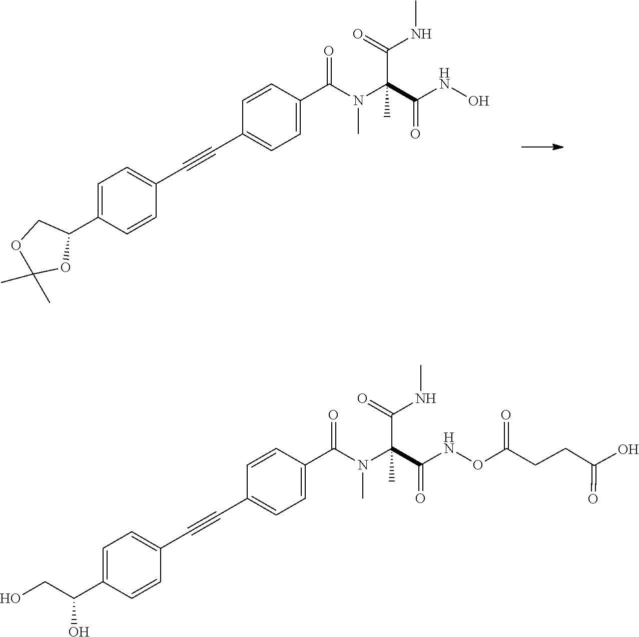 [SMILES: CNC(=O)[C@@](C)(C(=O)NO)N(C)C(=O)c1ccc(C#Cc2ccc([C@H]3COC(C)(C)O3)cc2)cc1 ; CNC(=O)[C@@](C)(C(=O)NOC(=O)CCC(=O)O)N(C)C(=O)c1ccc(C#Cc2ccc([C@H](O)CO)cc2)cc1]

Two milliliters (2 mL) of acetonitrile was added to 200 mg of (S)-2-(4-(4-(S)-2,2-dimethyl-1,3-dioxolan-4-yl)phenyl)ethynyl)-N-methylbenzamide)-N¹-hydroxy-N³,2-dimethylmalonamide, and 80 mg of succinic anhydride and 129 μL of pyridine were sequentially added, and the reaction mixture was stirred at room temperature for 5 and a half hours. Forty milligrams (40 mg) of succinic anhydride and 32 μL of pyridine were sequentially added to the reaction mixture and stirred overnight at room temperature. Ethyl acetate and water were added to the reaction mixture, and the pH was adjusted to 1.3 with 6 mol/L hydrochloric acid. The organic layer was separated, dried over anhydrous sodium sulfate, and the solvent was distilled off under reduced pressure. Three milliliters (3 mL) of tetrahydrofuran, 150 μL of water, and 15 mg of p-toluenesulfonic acid monohydrate were sequentially added to the resulting residue. The reaction mixture was stirred at room temperature for 3 hours and 30 minutes, and then 300 μL of water was added. The reaction mixture was stirred at room temperature for 1 hour and 30 minutes, and then 150 μL of water was added. The resulting mixture was allowed to stand overnight. Ethyl acetate and water were added to the reaction mixture, and the organic layer was separated. The obtained organic layer was dried over anhydrous sodium sulfate, the insoluble matter was filtered off, and the solvent was distilled off under reduced pressure. Ethyl acetate and IPE were added to the obtained residue, and the solid was collected by filtration, thereby obtaining 195 mg of 4-(((S)-2-(4-((4-((S)-1,2-dihydroxyethyl)phenyl)ethynyl)-N-methylbenzamide)-2-methy 1-3-(methylamino)-3-oxopropanamide)oxy)-4-oxobutanoic acid as a white solid.

¹H-NMR (400 MHz, CD₃OD) δ value: 1.84 (3H, s), 2.65-2.68 (2H, m), 2.76-2.82 (2H, m), 2.80 (3H, d, J=2.0Hz), 3.18 (3H, s), 3.61-3.63 (2H, m), 4.69-4.72 (1H, m), 7.41 (2H, d, J=8.2Hz), 7.51 (2H, d, J=8.2Hz), 7.56 (2H, d, J=8.6 Hz), 7.61 (2H, d, J=8.6 Hz); MS (ESI): 562[M+Na]⁺

The compound of Comparative Example 2 was unstable compared to the compound of Example 1, and was easily decomposed in 100 mmol/L tris-hydrochloric acid buffer (pH 7.4) to yield a compound A.

### Reference Example 2

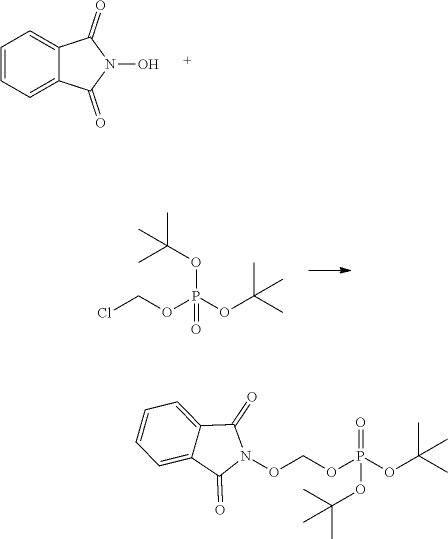 [SMILES: O=C1c2ccccc2C(=O)N1O ; CC(C)(C)OP(=O)(OCCl)OC(C)(C)C ; CC(C)(C)OP(=O)(OCON1C(=O)c2ccccc2C1=O)OC(C)(C)C]

Ten milliliters (10 mL) of N,N-dimethylformamide was added to 1 g of N-hydroxyphthalimide. The reaction mixture was ice-cooled, and 294 mg of 60% oily sodium hydride and 1.9 g of di-tert-butyl (chloromethyl)phosphate were sequentially added. The reaction mixture was stirred between 70° C. and 80° C. for 5 hours. Eighty-eight milligrams (88 mg) of 60% oily sodium hydride was added to the reaction mixture and stirred at 60° C. to 70° C. for 2 hours and 30 minutes. The reaction mixture was cooled, ethyl acetate and water were added, and the organic layer was separated. The organic layer was washed sequentially with an aqueous saturated sodium hydrogen carbonate solution, 1 mol/L hydrochloric acid, and an aqueous saturated sodium chloride solution. After drying by dehydration with anhydrous sodium sulfate, the solvent was distilled off under reduced pressure. The obtained residue was purified by silica gel column chromatography [eluent gradient:hexane:ethyl acetate=100:0 to 0:100], thereby obtaining 0.99 g of di-tert-butyl(((1,3-dioxoisoindoline-2-yl)oxy)methyl) phosphate as yellow oily matter.

¹H-NMR (400 MHz, CDCl₃) δ value: 1.44 (18H, s), 5.59 (2H, d, J=10.8 Hz), 7.72-7.80 (2H, m), 7.82-7.90 (2H, m)

### Reference Example 3

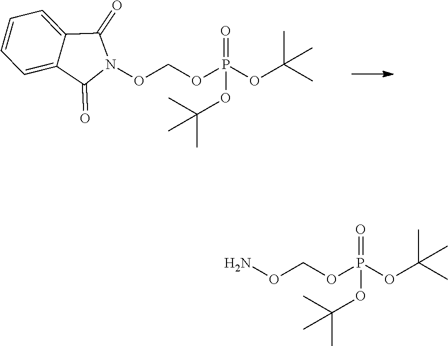 [SMILES: CC(C)(C)OP(=O)(OCON1C(=O)c2ccccc2C1=O)OC(C)(C)C ; CC(C)(C)OP(=O)(OCON)OC(C)(C)C]

Thirty milliliters (30 mL) of dichloromethane was added to 0.79 g of di-tert-butyl (((1,3-dioxoisoindoline-2-yl)oxy)methyl)phosphate. Under ice-cooling, 125 μL of methylhydrazine was added to the reaction mixture and stirred at the same temperature for 1 hour and 30 minutes. The solid was filtered off and the solvent was distilled off under reduced pressure. Five milliliters (5 mL) of dichloromethane was added to the residue, and the solid was filtered off. The solvent was distilled off under reduced pressure, thereby obtaining 692 mg of (aminooxy)methyl di-tert-butyl phosphate as yellow oily matter.

¹H-NMR (400 MHz, CD₃OD) δ value: 1.51 (18H, s), 5.17 (2H, d, J=11.2 Hz)

### Reference Example 4

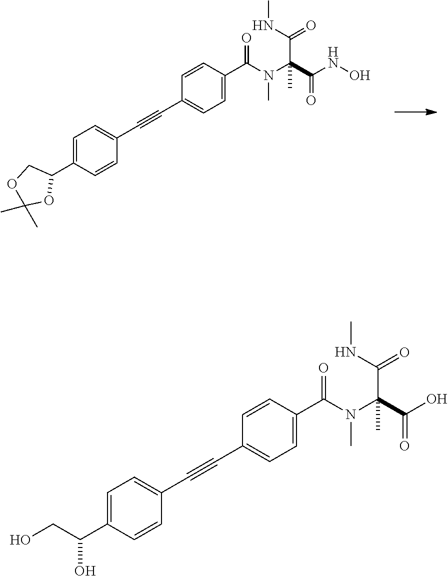

Eighteen milliliters (18 mL) of acetonitrile and 8.3 mL of 1 mol/L hydrochloric acid were added to 2.0 g of (S)-2-(4-((4-((S)-2,2-dimethyl-1,3-dioxolan-4-yl)phenyl)ethynyl)-N-methylbenzamide)-N¹-hydroxy-N³,2-dimethylmalonamide, and the reaction mixture was stirred overnight at room temperature. The reaction mixture was then stirred at 40° C. for 2 hours. After cooling the reaction mixture to room temperature, 9 mL of acetonitrile and 4.6 mL of 1 mol/L hydrochloric acid were added and stirred overnight at room temperature. Nine milliliters (9 mL) of acetonitrile and 8.3 mL of 1 mol/L hydrochloric acid were added to the reaction mixture and stirred at 40° C. for 4 hours and 30 minutes. The reaction mixture was cooled to room temperature and the solvent was distilled off under reduced pressure. Twenty milliliters (20 mL) of acetonitrile was added to the obtained mixture, the solvent was distilled off under reduced pressure, and water was azeotroped. The obtained residue was purified by silica gel column chromatography [eluent: chloroform:methanol=15:1], thereby obtaining 610 mg of (S)-2-(4-((4-((S)-1,2-dihydroxyethyl)phenyl)ethynyl)-N-methylbenzamide)-2-methyl-3-(methylamino)-3-oxopropanoic acid as a white solid.

¹H-NMR (400 MHz, DMSO-d₆) δ value: 1.66 (3H, s), 2.69 (2H, d, J=4.8 Hz), 3.02 (3H, s), 3.41-3.52 (2H, m), 4.57 (1H, t, J=6.0 Hz), 4.63-5.00 (1H, brs), 5.10-5.60 (1H, brs), 7.38-7.45 (2H, m), 7.48-7.60 (4H, m), 7.62-7.70 (2H, m), 8.40-8.50 (1H, m), 13.94 (1H, s)

### Reference Example 5

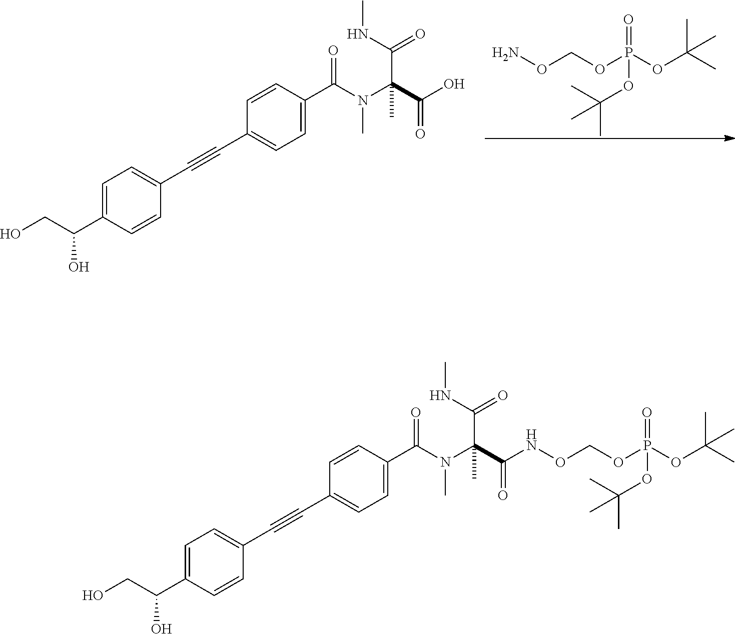 [SMILES: CNC(=O)[C@@](C)(C(=O)O)N(C)C(=O)c1ccc(C#Cc2ccc([C@H](O)CO)cc2)cc1 ; CC(C)(C)OP(=O)(OCON)OC(C)(C)C ; CNC(=O)[C@@](C)(C(=O)NOCOP(=O)(OC(C)(C)C)OC(C)(C)C)N(C)C(=O)c1ccc(C#Cc2ccc([C@H](O)CO)cc2)cc1]

Five hundred ninety-seven milligrams (597 mg) of (aminooxy)methyl di-tert-butyl phosphate, 20 mL of N,N-dimethylformamide, 181 μL of N,N-diisopropylethylamine, and 402 mg of O-(7-azabenzotriazole-1-yl)-N,N,N′,N′-tetramethyluronium hexafluorophosphate were added to 275 mg of (S)-2-(4-((4-((S)-1,2-dihydroxyethyl)phenyl)ethynyl)-N-methylbenzamide)-2-methyl-3-(methylamino)-3-oxopropanoic acid and stirred at room temperature for 3 hours and 30 minutes. Ethyl acetate and water were added to the reaction mixture, and the organic layer was separated. The organic layer was washed sequentially with a 10% aqueous citric acid solution and an aqueous saturated sodium chloride solution, and then dried over anhydrous sodium sulfate. After the solvent was distilled off under reduced pressure, the residue was purified by silica gel column chromatography [eluent: chloroform:methanol=80:20], thereby obtaining 210 mg of di-tert-butyl((((S)-2-(4-((4-((S)-1,2-dihydroxyethyl)phenyl)ethynyl)-N-methylbenzamid e)-2-methyl-3-(methylamino)-3-oxopropanamide)oxy)methyl) phosphate as colorless oily matter.

¹H-NMR (400 MHz, CDCl₃) δ value: 1.45 (9H, s), 1.51 (9H, s), 1.80 (3H, s), 2.87 (3H, d, J=4.8 Hz), 3.17 (3H, s), 3.58-3.85 (2H, m), 4.80-4.88 (1H, m), 5.31 (2H, d, J=14.8 Hz), 7.30-7.40 (2H, m), 7.48-7.65 (6H, m), 8.20-8.32 (1H, s), 11.34 (1H, s)

### Example 2

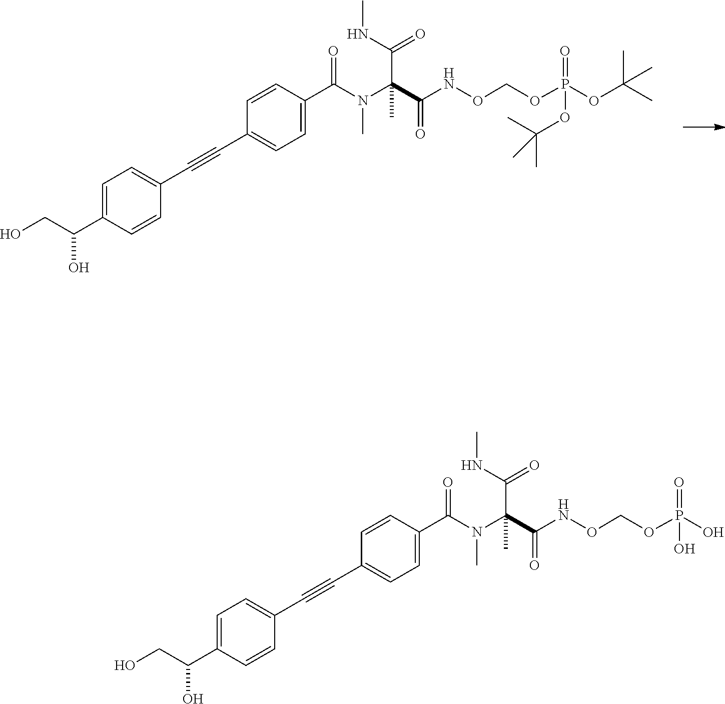 [SMILES: CNC(=O)[C@@](C)(C(=O)NOCOP(=O)(OC(C)(C)C)OC(C)(C)C)N(C)C(=O)c1ccc(C#Cc2ccc([C@H](O)CO)cc2)cc1 ; CNC(=O)[C@@](C)(C(=O)NOCOP(=O)(O)O)N(C)C(=O)c1ccc(C#Cc2ccc([C@H](O)CO)cc2)cc1]

Six milliliters (6 mL) of acetone and 6 mL of water were added to 210 mg of di-tert-butyl(((((S)-2-(4((4-((S)-1,2-dihydroxyethyl)phenyl)ethynyl)-N-methylbenzamid e)-2-methyl-3-(methylamino)-3-oxopropanamide)oxy)methyl) phosphate and stirred at 50° C. to 60° C. for 4 hours. Twenty milliliters (20 mL) of acetonitrile was added to the reaction mixture, and the mixture was concentrated under reduced pressure. Fifty-two microliters (52 μL) of 26% aqueous ammonia was added to the obtained mixture so as to adjust the pH to 7.8, and the mixture was purified by reverse phase silica gel column chromatography [eluent:acetonitrile:water=10:90]. The fractions containing the desired compound were collected and lyophilized, thereby obtaining 91 mg of (((S)-2-(4-((4-((S)-1,2-dihydroxyethyl)phenyl)ethynyl)-N-methylbenzamide)-2-methyl-3-(methylamino)-3-oxopropanamide)oxy)methyl dihydrogen phosphate as a white solid of the corresponding ammonium salt.

¹H-NMR (400 MHz, D₂O) δ value: 1.88 (3H, s), 2.83 (3H, s), 3.22 (3H, s), 3.73-3.84 (2H, m), 4.70-4.93 (1H, m), 5.08-5.30 (2H, m), 7.44-7.52 (2H, m), 7.57-7.77 (6H, m); MS (ESI): 548[M−H]⁻

### Examples 4 to 9

Vials were filled with 520 mg of a 10% aqueous solution of the compound of Example 1 and the additives listed in Table 2 below.

The vials were cooled to −60° C. and the contents were frozen. Thereafter, the shelf temperature was raised to −10° C. under vacuum (50 Pa or less), and primary drying was performed at the same pressure and temperature. After the product temperature reached −10° C. or higher, the shelf temperature was raised to 0° C., and secondary drying was performed at the same pressure and temperature. When the product temperature and the preset temperature almost coincided with each other and there was no change in the product temperature, drying was terminated, and the vials were sealed, thereby obtaining lyophilized preparations of Examples 4 to 9.

TABLE 2

| Example | Additive | Amount added |
|---|---|---|
| 4 | D-sorbitol | 16 mg |
| 5 | D-glucose | 16 mg |
| 6 | L-threonine | 11 mg |
| 7 | Citric acid monohydrate | 19 mg |
| 8 | Sodium chloride | 5 mg |
| 9 | — | — |

### Test Example 1 Solubility

The compounds of Examples 1 and 2 were taken in an amount of 1.2 mg, physiological saline was added during stirring until dissolution could be confirmed visually, and the solubility was calculated. The solubility of the compounds of Examples 1 and 2 was above 100 mg/mL.

The saturated solubility of the compound A as the drug substance was 0.2 mg/mL, and the compounds of Examples 1 and 2 had greatly improved water solubility compared to the compound A.

### Test Example 2 Multidrug-Resistant Pseudomonas aeruginosa Urinary Tract Infection Model Test in Mice

The mice used were ICR female SPF mice (5 weeks old: 5 mice per group). The inoculum solution was prepared by suspending a clinical isolate of Pseudomonas aeruginosa (S-2838 strain) cultured overnight at 37° C. on a Mueller-Hinton agar plate in sterile saline. The infection was induced by inoculating 0.2 mL (about 10³ CFU/mouse) of the inoculum solution into the urethra of each mouse. The test compound was dissolved in sterile saline and given once by tail vein administration 2 hours after infection. The count of viable cells in the kidney on the day after infection was recorded and the mean value was calculated.

As a result, compared with the control group to which the test compound was not administered, the count of viable cells in the kidney decreased by 4 log CFU/kidney or more in a group to which the compound of Example 1 was administered as a test compound at 12.5 mg/kg and a group to which the test compound of Example 2 was administered at 25 mg/kg. In the group to which the compound of Comparative Example 1 was administered as the test compound at 25 mg/kg, the count of viable cells in the kidney did not decrease by 4 log CFU/kidney or more as compared to the control group to which the test compound was not administered.

The compounds of Examples 1 and 2 exhibited excellent anti-Pseudomonas aeruginosa activity in the urinary tract infection model as compared to the compound of Comparative Example 1.

### Test Example 3 Storage Stability Test

The lyophilized formulations obtained in Examples 4 to 9 were stored at 25° C. or −20° C. for 1 month.

The purity of the compound of Example 1 after storage was measured by the HPLC method, and the residual rate was determined by the following equation. The results are shown in Table 3.

Residual rate (%)=(HPLC purity of compound A after storage/HPLC purity of compound A at the start of test)×100

### HPLC Measurement Conditions

### Detector: Liquid Chromatograph 1200 Series (Agilent Technology)

Measurement wavelength: 254 nm

### Column: XBridge C18 4.6×150 mm (Waters)

Column temperature: 40° C.
Flow rate: 1.0 mL/minute
Mobile phase A: water/(0.2 mol/L formate buffer (pH3))=90/10
Mobile phase B: acetonitrile/(0.2 mol/L formate buffer (pH3))=90/10
Gradient cycle: 0 min (Solution A/Solution B=90/10), 15 min (Solution A/Solution B =70/30), 20 min (Solution A/Solution B=0/100), 30 min (Solution A/Solution B=0/100)

TABLE 3

Residual rate

| Example | −20° C./1 month | 25° C./1 month |
|---|---|---|
| 4 | 99.8 | 97.7 |
| 5 | 99.2 | 97.0 |
| 6 | 98.3 | 93.7 |
| 7 | 99.2 | 95.7 |
| 8 | 98.3 | 88.9 |
| 9 | 97.1 | 85.2 |

The one-month residual rate at −20° C. of each lyophilized preparation was 97% or more, indicating good storage stability. In particular, the freeze-dried formulation of Example 4 to which a sugar alcohol was added had a residual rate of 97% or more at 25° C. for 1 month, and showed favorable storage stability.

### INDUSTRIAL APPLICABILITY

The compound of the present invention has strong antibacterial activity and excellent solubility in water, and thus, it is effective as a medicine.

1. A compound represented by the Formula [1] or a salt thereof: 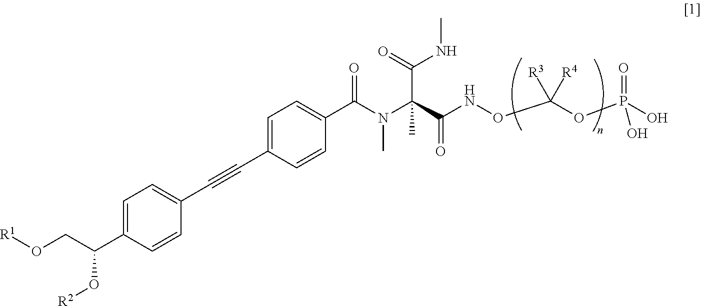
wherein R¹ represents a hydrogen atom, a group represented by the formula —PP(O)(OH)₂, or a hydroxyl protecting group, R² represents a hydrogen atom, a group represented by the formula —P(O)(OH)₂, or a hydroxyl protecting group, or R¹ and R² optionally form together an optionally substituted C₁₋₃ alkylene group or a group represented by the formula —P(O)(OH)—,
R³ represents a hydrogen atom or a C₁₋₆ alkyl group,
R⁴ represents a hydrogen atom or a C₁₋₆ alkyl group, and
n represents 0 or 1.

2. The compound or a salt thereof according to claim 1, wherein R¹ is a hydrogen atom.

3. The compound or a salt thereof according to claim 1, wherein R² is a hydrogen atom.

4. The compound or a salt thereof according to claim 1, wherein R³ is a hydrogen atom or a C₁₋₃ alkyl group.

5. The compound or a salt thereof according to claim 1, wherein R⁴ is a hydrogen atom or a C₁₋₃ alkyl group.

6. The compound or a salt thereof according to claim 1, wherein n is 0.

7. The compound or a salt thereof according to claim 1, wherein n is 1, R³ is a hydrogen atom or a methyl group, and R⁴ is a hydrogen atom.

8. A lyophilized formulation comprising the compound or a salt thereof according to claim 1.

9. An LpxC inhibitor comprising the compound or a salt thereof according to claim 1.

10. An antibacterial agent comprising the compound or a salt thereof according to claim 1.
